(19) United States
(12) Patent Application Publication
(10) Pub. No.: US 2026/0256550 A1
(43) Pub. Date: Sep. 3, 2026

(54) PERIODONTAL LIGAMENT REGENERATIVE MATERIAL

(71) Applicant: TOHOKU UNIVERSITY, Sendai-shi, Miyagi (JP)

(72) Inventors: Keiko MATSUI, Sendai-shi (JP); Shinji KAMAKURA, Sendai-shi (JP)

(21) Appl. No.: 19/163,698

(22) PCT Filed: Mar. 15, 2023
(86) PCT No.: PCT/JP2023/010116

§ 371 Date: Sep. 9, 2025

(51) Int. Cl. A61C 8/02; A61K 6/58; A61K 6/69

(52) U.S. Cl. CPC A61C 8/0006; A61K 6/58; A61K 6/69

(57) ABSTRACT

A periodontal ligament regenerative material including a composite containing octacalcium phosphate and collagen, wherein the composite is a porous body having a large number of pores, and the porous body has a porosity of 80% or more and 98% or less, a Young's modulus in a dry state of 0.1 MPa or more and 20.0 MPa or less, and a Young's modulus in a wet state of 0.05 MPa or more and 5.0 MPa or less.

### TECHNICAL FIELD

The present invention relates to a periodontal ligament regenerative material.

### BACKGROUND ART

Because self-healing process is difficult for advanced periodontal disease accompanied by extensive destruction of periodontal tissues, it is required a treatment associated with periodontal tissue regeneration. In particular, the periodontal ligament, which is situated between the alveolar bone and the tooth, is mainly composed of fibrous connective tissue that connects the surface (cementum) of the tooth root and the alveolar bone, and it is an important tissue that absorbs and relieves the force applied to the tooth when chewing food, and acts as a cushion that alleviates direct transmission of the force applied to the tooth to the alveolar bone. Therefore, it has been proposed various regenerative therapy for periodontal ligament. The healing of the tooth root surface obtained by periodontal surgical therapy prior to the regenerative therapy of periodontal ligament is a non-physiological “epithelial attachment” due to proliferation of epithelial cells. In contrast, the regenerative therapy of periodontal ligament aims at “connective tissue attachment”, which is physiological repair.

For example, guided tissue regeneration (GTR) is used to seek to regenerate bone which is destroyed by bacteria, and it is clinically applied by inserting an artificial material such as a biocompatible Gore-Tex (registered trademark) membrane or a polylactic acid membrane is inserted between existing bone and tooth, which is considered to prevent an unnecessary tissue from entering a healing region.

As shown in Patent Document 1, it is generally accepted that the artificial material alone has a limited regenerative ability. Therefore, it is already clinically applied for affected tooth root surface to regenerate periodontal tissue by combination with an artificial material and a physiological active substance such as enamel matrix or basic fibroblast growth factor (FGF-2) which acts on cell proliferation and differentiation. And, there is a problem that these agents cannot be denied to proliferate malignant cell, when malignant tumor is in the surgical site or their vicinity, because it aims to regenerate by cell proliferation mechanism. Therefore, it is hard to say that a standard method of periodontal tissue regeneration has been established.

### CITATION LIST

### Patent Documents

Patent Document 1: JP 2014-230685 A
Patent Document 2: JP 5046511 B
Patent Document 3: JP 2021-115165 A
Patent Document 4: JP 2010-035834 A
Patent Document 5: JP 6094716 B

### SUMMARY OF INVENTION

### Technical Problem

As shown in Patent Documents 2 and 3, it has been conventionally known that octacalcium phosphate (OCP) collagen composite (OCP/Col) is effective for regeneration of bone, which is a hard tissue. As shown in Patent Document 4, it is known that an artificial bone material that composed of biodegradable polymer and calcium phosphate, which is a precursor of hydroxyapatite, is effective for cortical bone regeneration, such as the alveolar bone in the dental field and the long bone in the orthopedic field. As shown in Patent Document 5, it is known that a porous composite containing OCP and teriparatide is effective for regeneration of bone, which is a hard tissue. However, it has never been known those effective for soft tissue regeneration, such as periodontal ligament.

The present invention has been made in view of the above circumstances, and an object thereof is to provide a periodontal ligament regenerative material which is enable to regenerate a periodontal ligament alone at a site where periodontal tissue regeneration is required.

### Solution to Problem

As a result of intensive studies, the present inventors have surprisingly obtained the following findings. The regeneration of periodontal ligament is accomplished when octacalcium phosphate (hereinafter referred to as “OCP”) collagen composite (hereinafter referred to as “OCP/Col”), which is known to be effective for regeneration of bone as a hard tissue, is implanted to bone defect around a tooth to expect a physiological natural healing and recovery.

The present invention has the following aspects.

[1] A periodontal ligament regenerative material including a composite containing OCP and collagen, in which
the composite is a porous body having a large number of pores, and
the porous body has a porosity of 80% or more and 98% or less, a Young's modulus in a dry state of 0.1 MPa or more and 20.0 MPa or less, and a Young's modulus in a wet state of 0.05 MPa or more and 5.0 MPa or less.

[2] The periodontal ligament regenerative material according to [1], in which the OCP and the collagen are blended at a mass ratio of from 0.5 to 35 of the OCP to 1 of the collagen.

[3] The periodontal ligament regenerative material according to [1] or [2], including a cytokine having an osteogenic ability.

[4] The periodontal ligament regenerative material according to any one of [1] to [3], in which the composite is obtained by immersing a gel, a sol, or a liquid containing the OCP and the collagen in a liquid refrigerant having a temperature lower than a freezing temperature of the gel, the sol, or the liquid, to pre-freeze the gel, the sol, or the liquid, and then freeze-drying the gel, the sol, or the liquid.

[5] The periodontal ligament regenerative material according to any one of [1] to [4], in which the composite is subjected to a heat treatment at a temperature of 50° C. or higher and 200° C. or lower and a pressure of 0 Pa or more and 3000 Pa or less for a treatment time of 0.1 days or longer and 10 days or shorter.

### Advantageous Effects of Invention

According to the present invention, a periodontal ligament regenerative material can provide a capability of regenerating a periodontal ligament alone at a site where periodontal tissue regeneration is required.

### BRIEF DESCRIPTION OF DRAWINGS

FIG. 1 is a diagram illustrating a mandibular third premolar (P3) and a mandibular fourth premolar (P4) exposed in an Example.

FIG. 2 is an X-ray photograph before a bone defect is created in the Example.

FIG. 3 is an X-ray photograph after the bone defect is created in the Example.

FIG. 4 is an X-ray photograph immediately after the octacalcium phosphate/collagen composite (OCP/Col) discs are implanted into the bone defect in the Example.

FIG. 5 is an X-ray photograph after one month of OCP/Col discs implantation at the bone defect in the Example.

FIG. 6 is an X-ray photograph after two months of OCP/Col discs implantation at the bone defect in the Example.

FIG. 7 is an X-ray photograph after three months of OCP/Col discs implantation at the bone defect in the Example

FIG. 8 is a Micro-CT photograph after three months of OCP/Col discs implantation at the bone defect in the Example.

FIG. 9 is a Micro-CT photograph after three months of untreated group in the Example.

FIG. 10 is a specimen of micrograph prepared in the Example.

FIG. 11 is a micrograph in which a part of FIG. 10 is enlarged.

### DESCRIPTION OF EMBODIMENTS

An embodiment of the periodontal ligament regenerative material of the present invention will be described.

The present embodiments are specifically described in order to make the gist of the invention better understandable, and unless particularly stated otherwise, the embodiments are not intended to limit the present invention.

### Periodontal Ligament Regenerative Material

The periodontal ligament regenerative material of the present embodiment includes a composite containing octacalcium phosphate (hereinafter referred to as “OCP”) and collagen.

In the periodontal ligament regenerative material of the present embodiment, the composite is a porous body having a large number of pores.

The porous body has a porosity of 80% or more and 98% or less, preferably 85% or more and 90% or less, and more preferably 85% or more and 90% or less. If the porosity of the composite is less than the lower limit value, periodontal tissue regeneration is not sufficiently performed. If the porosity of the composite exceeds the upper limit value, handling performance is deteriorated, and it decreases the accuracy of implantation to the designated site.

The porosity of the porous body is determined by the following formula (1) using a total pore volume and an apparent density by mercury porosimetry.

$$Porosity ( \% ) = total pore volume / \{ ( 1 / apparent density ) + total pore volume \} \times 100 \qquad ( 1 )$$

The porous body has a Young's modulus in a dry state of 0.1 MPa or more and 20.0 MPa or less, preferably 0.5 MPa or more and 16.0 MPa or less, and more preferably 0.8 MPa or more and 10.0 MPa or less. When the Young's modulus in a dry state of the porous body is less than the lower limit value, periodontal tissue regeneration is not sufficiently performed. If the Young's modulus in a dry state of the porous body exceeds the upper limit value, handling performance is deteriorated, and it decreases the accuracy of implantation to the designated site.

The dry state of the porous body refers to a state in which the composite prepared through a freeze-drying treatment does not contain moisture.

The porous body has a Young's modulus in a wet state of 0.05 MPa or more and 5.0 MPa or less, preferably 0.07 MPa or more and 2.0 MPa or less, and more preferably 0.1 MPa or more and 1.0 MPa or less. When the Young's modulus in a wet state of the porous body is less than the lower limit value, periodontal tissue regeneration is not sufficiently performed. If the Young's modulus in a wet state of the porous body exceeds the upper limit value, handling performance is deteriorated, and it decreases the accuracy of implantation to the designated site.

The wet state of the porous body refers to a state in which 0.5 mL of physiological saline is dropped on a sample (10 mm×10 mm×10 mm) in consideration of exposure of the sample to blood in a clinical situation. The Young's modulus in a wet state of the porous body starts to be measured within 5 minutes after the physiological saline is dropped.

The Young's moduli of the porous body are measured using a compression tester (EZ-L 5 kN, available from Shimadzu Corporation). A sample molded to a predetermined size (10 mm×10 mm×10 mm) is compressed at a cross head speed of 0.5 mm/min for a sample time of 50 msec under a maximum load of 4500 N. In the wet condition, the measurement is started within 5 minutes after 0.5 mL of the physiological saline is dropped on the sample. From the obtained load-displacement curve, a stress-strain curve is obtained, and the Young's modulus of the sample is calculated.

A pore size of the porous body is preferably 3 μm or more and 90 μm or less, more preferably 5 μm or more and 40 μm or less, and still more preferably 7 μm or more and 36 μm or less. When the pore size is less than the lower limit value, invasion of periodontal tissue-forming cells is limited, and the effect of periodontal tissue regeneration may be reduced. When the pore size exceeds the upper limit value, mechanical strength of the porous body tends to decrease.

The pore size of the porous body is measured by using a pore distribution measurement with a mercury porosimeter. Specifically, the measurement is performed by the following method.

### Pore Size Measurement

As a pretreatment, the sample (porous body) is dried at a constant temperature of 120° C. for 4 hours. For each sample after the pretreatment, a pore distribution of pore sizes of from 0.0018 μm to 200 μm is determined under the following conditions by the mercury porosimetry using the following measurement apparatus.
Measurement apparatus: Autopore IV9520 (available from Micromeritics)
Measuring condition: Contact angle between sample and mercury: 140 deg
Surface tension of mercury: 0.48 N/m (converted as 1 dyne=10⁻⁵ N)

In the pore distribution curve obtained by the mercury porosimetry, the value of the pore size (diameter) showing the maximum value of the peak having the largest area is the value of the pore size in the present embodiment.

In the porous body, a proportion of pores having a pore size of 71 μm or more and 200 μm or less in total pores having a pore size of 200 μm or less in the pore distribution measured by the mercury porosimetry is preferably 8% or less, and more preferably 3% or more and 8% or less. The proportion of the pores is represented by the following formula (2) using the cumulative pore volume and total pore volume measured by the mercury porosimeter.

$$Pore proportion ( \% ) = cumulative pore volume / total pore volume \times 100 \qquad ( 2 )$$

The shape of the porous body can be arbitrarily set in consideration of the shape, size, and the like of the affected part to be filled with the porous body. For example, the porous body is preferably in the shape of a rectangular parallelepiped (block body), a cube, a cylinder, a tablet, or a granule.

When the porous body is rectangular parallelepipedal, the size thereof is preferably 1 mm×1 mm×1 mm or more, and the upper limit is preferably 20 mm×20 mm×20 mm.

When the porous body is cylindrical, the size thereof is preferably 1 mm or more and 20 mm or less, and the height thereof is preferably 1 mm or more and 20 mm or less.

In the periodontal ligament regenerative material of the present embodiment, when the porous body is granular, the shape is not limited to a sphere and may be an indefinite shape, but the diameter is preferably 0.1 mm or more and 10 mm or less. The diameter of the granular porous body is determined by a sieving test method.

OCP is a publicly known material and can be prepared by, for example, the dropping method of LeGeros (LeGeros R Z, Calcif Tissue Int 37: 194-197, 1985), or the method using a synthesizer (three way tube) described in JP 3115642 B.

The origin, properties, and the like of collagen are not particularly limited, and it can be used various types of collagens. As collagen, it is preferable to use an enzyme-solubilized collagen, which is obtained by solubilization by a proteolytic enzyme (e.g., pepsin and pronase), with removal of telopeptide. From the viewpoint of materials science, the type of collagen is preferable type I (or type I+type III) collagen derived from the skin, bone, tendon, etc. of pigs, cows, etc. Because collagen is a component derived from a living body, it has high safety, and the enzyme-solubilized collagen is especially preferable by its low allergenicity.

As collagen, it may be used a commercially available product.

In the periodontal ligament regenerative material of the present embodiment, the blending ratio of OCP to collagen can be appropriately adjusted to the desired shape, handling, biocompatibility, etc. OCP and collagen are blended at a mass ratio of preferably 0.5 or more and 35 or less, more preferably 1 or more and 20 or less, still more preferably 2 or more and 10 or less, and most preferably 3 or more and 5 or less of OCP to 1 of collagen. When the blending ratio is less than the lower limit value, it may be deteriorated the function of periodontal tissue regeneration of the obtained composite. When the blending ratio is more than the upper limit value, it may be deteriorated the property of moldability.

The periodontal ligament regenerative material of the present embodiment may include osteogenic cytokines (for example, bone morphogenetic protein-2, and transforming growth factor β 1) in addition to OCP and collagen. The inclusion of the cytokine can increase a rate of periodontal tissue regeneration.

A content of the cytokine in the periodontal ligament regenerative material of the present embodiment can be appropriately adjusted to the desired rate of periodontal tissue regeneration, and so on.

The periodontal ligament regenerative material of the present embodiment may include teriparatide corresponding to the N-terminal 34 amino acid region of wild-type parathyroid hormone (PTH). The teriparatide is sold as a therapeutic agent for osteoporosis under the name Forteo® or Teribone®.

The periodontal ligament regenerative material of the present embodiment may include a component commonly used in this field. Examples of such a component include bioabsorbable polymers (e.g., polylactic acid and polylactic acid-polyethylene glycol copolymer), bioabsorbable calcium phosphates (e.g., β-TCP), and non-bioabsorbable materials (e.g., HA and ceramics).

It is not particularly limited the implantation manner of the periodontal ligament regenerative material of the present embodiment. For example, the implantation of the periodontal ligament regenerative material is performed by filling a site where periodontal tissue regeneration, particularly periodontal ligament regeneration is required, with an appropriate size of the periodontal ligament regenerative material. When a sufficient amount of blood or body fluid is present at a site where periodontal tissue regeneration is required (for example, defect of periodontal tissue), the periodontal ligament regenerative material can be used as it is or cut into an appropriate shape and used for filling. When it is not found a sufficient amount of blood or the like at the implantation site, or it cannot be filled the periodontal ligament regenerative material in the original shape, the periodontal ligament regenerative material is immersed in blood, physiological saline, and so on, thereafter it can be filled the periodontal tissue defect after confirmation of its sponge-like elasticity.

The periodontal tissue is composed of cementum, gum (gingiva), periodontal ligament (periodontal membrane), alveolar bone, etc. Periodontal tissue defects can be caused by various factors. Examples of the factor include dental caries (tooth decay) and periodontal diseases.

As regenerative treatment of periodontal tissue, it has traditionally been performed a non-surgical treatment of non-progressive case and a surgical treatment of progressive case.

As the surgical treatment, it includes flap surgery, soft tissue transplantation, bone graft (bone transplantation), guided tissue regeneration, local application of tissue-stimulating proteins, and so on.

Application of the periodontal ligament regenerative material of the present embodiment enables alone to regenerate periodontal ligament, when it is implanted to the designated site.

Furthermore, the following problems are encountered the regenerative treatment of periodontal tissue in the present time. When the cementum is removed and the exposed dentin is targeted for treatment, the regeneration of the cementum does not occur spontaneously. In the absence of cementum regeneration, epithelial attachment of the dentin surface and epithelial cells occurs, but is much weaker than cementum-mediated connective tissue attachment. Further, a method for regenerating the cementum alone has not been established so far. According to the periodontal ligament regenerative material of the present embodiment, the cementum can also be regenerated alone when it is implanted to the designated site.

In addition, according to the periodontal ligament regenerative material of the present embodiment, the periodontal tissue can be cultured by administering the periodontal ligament regenerative material to a tissue piece of the periodontal tissue.

The subject is not particularly limited, and examples thereof include animals such as humans, dogs, cats, monkeys, and rats. In the present embodiment, a preferred subject is a human.

### Method for Preparing Periodontal Ligament Regenerative Material

The method for preparing the periodontal ligament regenerative material of the present embodiment is not particularly limited, and various methods can be used as long as they are methods for preparing a composite containing OCP and collagen. A preferred method for preparing the periodontal ligament regenerative material of the present embodiment includes, for example, the following preparation methods.

### (a) Method of Mixing OCP to Form Composite

The pH of a collagen solution having an appropriate concentration is adjusted to a range where gelation can occur, and OCP is added to the collagen solution, followed by sufficient kneading to prepare a mixture of OCP and collagen. Then, the mixture is freeze-dried to obtain a composite powder. The composite powder is molded by a suitable template to prepare a molded product, which is then sterilized by a commonly used sterilization method (e.g., electron beam irradiation or high-pressure steam sterilization).

### (b) Method of Mixing OCP Suspension to Form Composite

The pH of an acidic collagen solution having an appropriate concentration is aseptically adjusted to from 5.5 to 7.5 with an appropriate buffer (e.g., phosphate buffer, Tris buffer, or sodium acetate buffer), and OCP is added before collagen is gelled to prepare a suspension of collagen and OCP. Subsequently, the suspension is poured into a mold to give a shape, and then gelatinized at an appropriate temperature (for example, 37° C.), when the pH is maintained neutral to weakly alkaline. Thereafter, the obtained gel form a composite, when it is repeatedly washed with water to remove the salt and the like in the buffer, and the composite is sterilized in the same manner as described above.

Alternatively, the suspension, which is gelatinized and washed with water and eliminates the procedures of molding, is freeze-dried and sterilized in the same manner as described above, and can apply to the affected site as an amorphous filling material.

### (c) Method of Precipitating OCP on Collagen to Form Composite

The pH of an acidic collagen solution having an appropriate concentration is aseptically adjusted to from 5.5 to 7.5 with an appropriate buffer (e.g., phosphate buffer, Tris buffer, or sodium acetate buffer), and a calcium solution and a phosphoric acid solution are added thereto before collagen is gelled, thereby precipitating OCP on collagen. Thereafter, the solution is poured into a mold to give a shape, and then gelled at an appropriate temperature (for example, 37° C.), when the pH is maintained neutral to weakly alkaline, and then washed with water repeatedly to remove the salt in the buffer and the like, thereby obtaining a composite, which is then sterilized in the same manner as described above.

Alternatively, the solution, which is gelled and eliminates the procedures of molding, is freeze-dried and sterilized in the same manner as described above, and can apply to the affected site as an amorphous filling material.

The precipitation of OCP on collagen is now described. The precipitation of the OCP is based on a degree of supersaturation (ion product/solubility product) determined by Ca²⁺, PO³⁻, pH, and the like. Therefore, a Ca²⁺ solution and a PO³⁻ solution are poured into a collagen solution whose pH has been adjusted, under the condition that the solution is supersaturated with respect to OCP, and the OCP is precipitated. OCP precipitates either spontaneously in the collagen interstices or on the surfaces of the collagen fibers using them as nuclei. The OCP is known to be converted to OCP via dicalcium phosphate (DCP or its dihydrate DCPD) or amorphous calcium phosphate (ACP), and to be finally matured to HA. The particle size of the OCP is preferably adjusted to 10 μm or more and 1000 μm or less, more preferably adjusted to 100 μm or more and 500 μm or less, and still more preferably adjusted to 300 μm or more and 500 μm or less. The particle size of the OCP can be classified by the size of the sieve mesh.

In the present embodiment, the method for obtaining a porous composite preferably includes immersing a gel, a sol, or a liquid containing OCP and collagen in a liquid refrigerant, to freeze (pre-freeze) the gel, the sol, or the liquid, and then freeze-drying the gel, the sol, or the liquid. The phrase “immersing a gel, a sol, or a liquid in a liquid refrigerant, to freeze the gel, the sol, or the liquid” also includes, for example, an aspect in which a container containing a gel, a sol, or a liquid is sealed and then immersed in a liquid refrigerant to freeze the gel, the sol, or the liquid.

The liquid refrigerant is a liquid having a temperature lower than the freezing temperature of the gel, the sol, or the liquid containing OCP and collagen, and examples thereof include methanol, ethanol, acetone, acetonitrile, and liquid nitrogen. The temperature of the liquid refrigerant is, for example, −20° C. or lower, preferably −40° C. or lower, −60° C. or lower, −80° C. or lower, −100° C. or lower, −120° C. or lower, −140° C. or lower, −160° C. or lower, or −180° C. or lower. It is considered that the pore size of the resulting porous composite can be reduced by immersing the gel, the sol, or the liquid containing OCP and collagen in the liquid refrigerant to rapidly freeze the gel, the sol, or the liquid.

The composite is preferably subjected to a heat treatment. By the heat treatment, a part of the OCP molecular structure is collapsed to enable to invade periodontal tissue forming cells, and consequently regeneration of periodontal tissue is promoted and crosslinked collagen causes to improve shape retention.

The temperature of the heat treatment (heat treatment temperature) is preferably 50° C. or higher and 200° C. or lower, more preferably 60° C. or higher and 180° C. or lower, and still more preferably 95° C. or higher and 150° C. or lower.

The pressure of the heat treatment is preferably 0 or more and 3000 Pa or less, and more preferably 0 or more and 300 Pa or less.

The time of the heat treatment (heat treatment time) is preferably 0.1 days or longer and 10 days or shorter, and more preferably 0.5 days or longer and 5 days or shorter.

### EXAMPLES

The present invention will be described in detail with reference to the following examples, but the present invention is not limited to the examples.

The OCP and collagen used are as follows.

### (1) OCP

OCP is synthesized based on the synthesizer (three way tube) described in JP 3115642 B, dried and consolidated, crushed, then sieved in a range of from 300 to 500 μm, and sterilized with dry heat at 120° C. for 2 hours was used.

The method for producing OCP will be described in detail.

Three liquids, i.e., an aqueous solution containing 0.16 mol of calcium acetate (Ca(CH₃COO)₂·H₂O) in a total amount of 1500 mL, an aqueous solution containing 0.16 mol of sodium dihydrogen phosphate dihydrate in a total amount of 1500 mL, and 1000 mL of pure water, were warmed to 70° C., and introduced from each supply port of a three way tube in 15 minutes using an apparatus having three supply ports (three way tube). The obtained precipitate was aged at 70° C. for 15 minutes, then filtered, dispersed in pure water again, filtered, then sized, and dried.

### (2) Collagen

A freeze-dried powder of pepsin-digested atelocollagen derived from the porcine dermis (type I+type III, neutral, crosslinked: NMP collagen PS; available from NH Foods Ltd.) was used.

A collagen tablet was prepared by dissolving the collagen, adjusting the final concentration to 3% and the pH to 7.4, freeze-drying the collagen solution, molding the solution into discs of 9 mm in diameter and 1.5 mm in thickness, and sterilizing the tablet by electron beam irradiation (15 kGy).

### Example

### Preparation of OCP-Collagen Composite

The collagen was dissolved and adjusted to have a final concentration of 3% and a pH of 7.4, and 10% (w/w) OCP (from 300 to 500 μm in diameter) was added to the collagen solution and thoroughly mixed. The OCP-collagen mixture was freeze-dried, shaped into discs of 9 mm in diameter and 1.5 mm in thickness, heated at 150° C. for 24 hours under reduced pressure to perform dehydrothermal crosslinking, and sterilized by electron beam irradiation (15 kGy) (the obtained OCP-collagen composite is hereinafter referred to as “OCP/Col”).

The moldability of OCP/Col was very good, and the molded product could be easily prepared. The OCP and collagen were contained in the composite in amounts of about 10.5 mg and about 3.5 mg, respectively, and the porosity and Young's modulus of the composite were about 93% and about 2.3 MPa, respectively.

### Test Example

The OCP/Col prepared in the Example was implanted into a bone defect, and its function was tested. Specific methods, results, and the like are as follows.

### (1) Materials and Methods

Experimental animal: Adult beagle dogs were used.

### (2) Surgical Operation

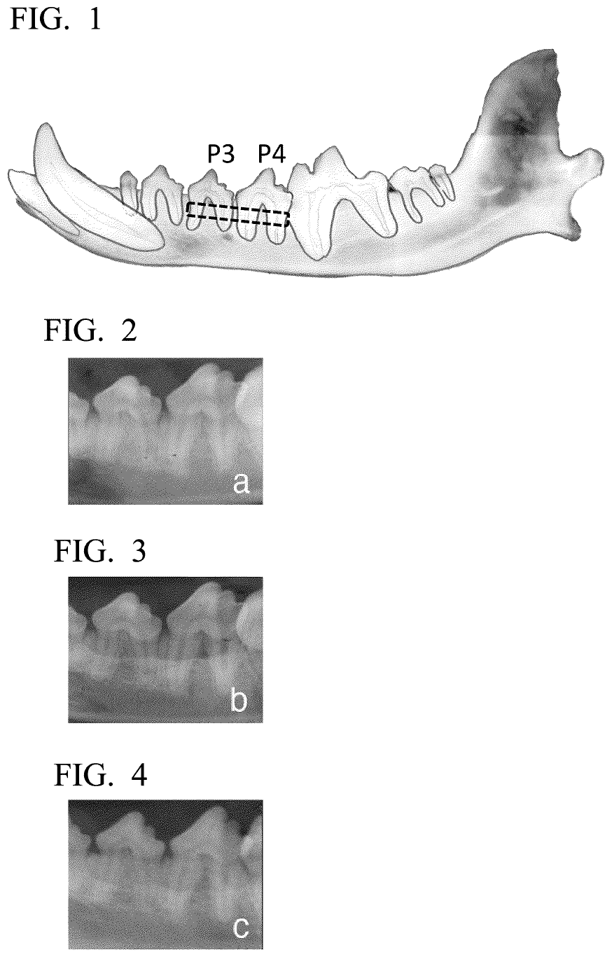

A bone defect was created in the buccal tooth root part of the mandibular premolar of a beagle dog, and an OCP/Col disc (9 mm in diameter, 1.5 mm in thickness) was implanted. Specifically, the mucoperiosteum was ablated through a lateral skin incision to expose the mandible. The attached gingiva of approximately 5 mm from the alveolar crest was not ablated and was preserved. The exposed bones of the tooth root parts of the mandibular third premolar (P3 shown in FIG. 1) and the mandibular fourth premolar (P4 shown in FIG. 1) were removed until it reached the root surface. In this manner, a band-shaped bone defect was created in a width of about 3 mm×a length of from 25 to 30 mm. After that, the OCP/Col discs were implanted into the created bone defect.

As a comparative control, an untreated group: a group in which only a bone defect was created and the bone defect was closed without implanting anything was used.

### (3) Experiment Period

The period of implantation of the sample was 3 months, and 5 experimental animals were used in each group and each period.

### (4) Intraoral X-Ray Photography

Intraoral radiography was performed immediately after OCP/Col implantation, one month after implantation, two months after implantation, and three months after implantation.

For the X-ray photography, a portable radiography apparatus (PORT-XIII: MORITA) and an instant film (D-sensitivity: standard; Hanshin Technical Laboratory, LTD.) were used, and the X-ray photography was performed under the conditions of 60 kv, 2 mA, and 0.8 seconds, followed by predetermined development process.

### (5) Micro-CT Photography

Micro-CT photography was performed on the specimens excised three months after implantation of the OCP/Col discs.

The Micro-CT photography was performed using an X-ray CT apparatus for experimental animals (Latheta LCT-200; Hitachi Aloka Medical, Ltd.) under the conditions of 50 kVp, 500 μA, and 3.6 ms, with a slice size of 240 μm, a slice thickness of 240 μm, and a pixel size of 120 μm.

### (6) Specimen Preparation Method

After the 3-month follow-up period, the animals were euthanized (secobarbital was intravenously administered in a large amount after intramuscular injection of ketaral and a mixed solution of medetomidine hydrochloride and midazolam), and then the mandible was excised and fixed by immersion in 10% neutral buffered formalin.

Thereafter, the mandible was decalcified with 10% EDTA or citric formate, and prepared paraffin sections were subjected to hematoxylin-eosin staining and azan staining for histological evaluation.

### Results

### (1) Intraoral X-Ray Photography

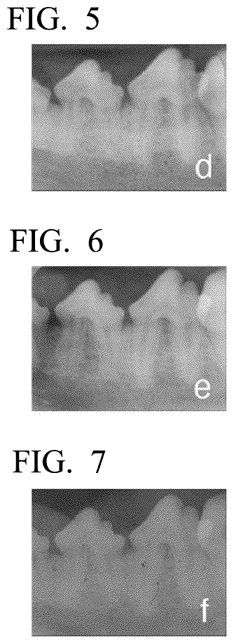

The intraoral radiography is shown in FIGS. 2 to 7. FIG. 2 is an intraoral radiography before the bone defect creation in the Example. FIG. 3 is an intraoral radiography after the bone defect creation in the Example. FIG. 4 is an intraoral radiography immediately after implantation of the OCP/Col discs into the bone defect in the Example. FIG. 5 is an intraoral radiography of one month after implantation of the OCP/Col discs into the bone defect in the Example. FIG. 6 is an intraoral radiography of two months after implantation of the OCP/Col discs into the bone defect in the Example. FIG. 7 is an intraoral radiography of three months after implantation of the OCP/Col discs into the bone defect in the Example. From the results shown in FIGS. 2 to 7, it was confirmed that the regeneration of periodontal tissue progressed due to implantation of the OCP/Col discs into the bone defect.

### (2) Micro-CT Photography

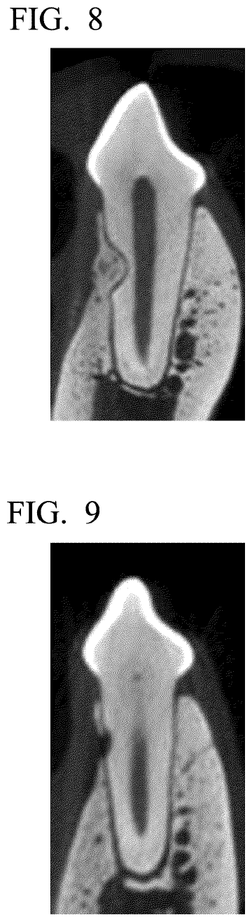

The Micro-CT photography of the oral cavity is shown in FIGS. 8 and 9. FIG. 8 is a Micro-CT photograph of three months after implantation of the OCP/Col discs into the bone defect in the Example. FIG. 9 is a Micro-CT photograph of the untreated group after three months in the Example. From the results shown in FIG. 8, it was confirmed that the regeneration of periodontal tissue progressed due to implantation of the OCP/Col discs into the bone defect. On the other hand, it was confirmed that the regeneration of periodontal tissue did not progress in the untreated group.

### (3) Histological Findings

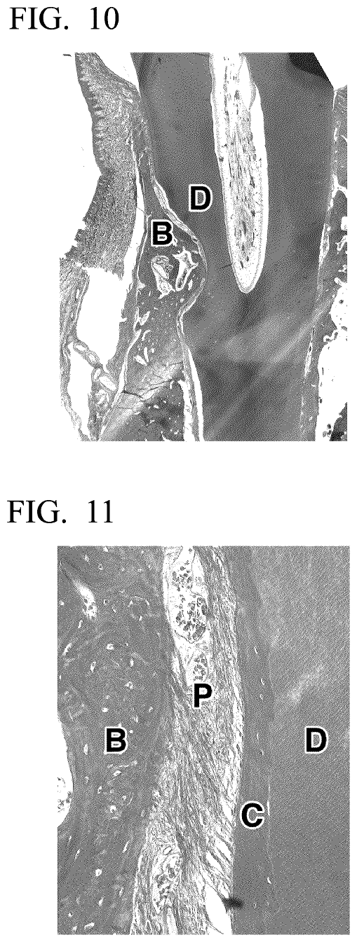

The histological findings (azan staining) of the specimens are shown in FIGS. 10 and 11. FIG. 10 is a micrograph of the specimen prepared in the Example. FIG. 11 is a micrograph in which a part of FIG. 10 is enlarged.

From the results shown in FIG. 10, it was confirmed that the periodontal ligament was regenerated between the regenerated alveolar bone (B) and dentin (D) by implantation of the OCP/Col discs. In addition, from the results shown in FIG. 11, it was confirmed that cementum (C) was regenerated on the surface of the depressed dentin (D), and that a periodontal ligament (P) was regenerated so as to connect the cementum (C) and the alveolar bone (B).

1. A periodontal ligament regenerative material comprising a composite containing octacalcium phosphate and collagen, wherein
the composite is a porous body having a large number of pores, and
the porous body has a porosity of 80% or more and 98% or less, a Young's modulus in a dry state of 0.1 MPa or more and 20.0 MPa or less, and a Young's modulus in a wet state of 0.05 MPa or more and 5.0 MPa or less.

2. The periodontal ligament regenerative material according to claim 1, wherein the octacalcium phosphate and the collagen are blended at a mass ratio of from 0.5 to 35 of the octacalcium phosphate to 1 of the collagen.

3. The periodontal ligament regenerative material according to claim 1, comprising a cytokine having an osteogenic ability.

4. The periodontal ligament regenerative material according to claim 1, wherein the composite is obtained by immersing a gel, a sol, or a liquid containing the octacalcium phosphate and the collagen in a liquid refrigerant having a temperature lower than a freezing temperature of the gel, the sol, or the liquid, to pre-freeze the gel, the sol, or the liquid, and then freeze-drying the gel, the sol, or the liquid.

5. The periodontal ligament regenerative material according to claim 1, wherein the composite is subjected to a heat treatment at a temperature of 50° C. or higher and 200° C. or lower and a pressure of 0 Pa or more and 3000 Pa or less for a treatment time of 0.1 days or longer and 10 days or shorter.
